(19) United States
(12) Patent Application Publication
(10) Pub. No.: US 2021/0259988 A1
(43) Pub. Date: Aug. 26, 2021

(54) ELECTROSPUN STRUCTURES HAVING A PHARMACEUTICAL AND METHODS OF MAKING AND USING THE SAME

(71) Applicant: NANOFIBER SOLUTIONS, LLC, Dublin, OH (US)

(72) Inventors: Jed K. JOHNSON, London, OH (US); Katie KAFFENBARGER, Dublin, OH (US); Devan OHST, Columbus, OH (US); Ross KAYUHA, Dublin, OH (US)

(73) Assignee: NANOFIBER SOLUTIONS, LLC, Dublin, OH (US)

(21) Appl. No.: 17/181,671

(22) Filed: Feb. 22, 2021

Related U.S. Application Data
(60) Provisional application No. 62/979,818, filed on Feb. 21, 2020.

(51) Int. Cl. A61K 9/70; A61K 31/05; A61K 47/34

(52) U.S. Cl. CPC A61K 9/7007; A61K 9/006; A61K 47/34; A61K 31/05

(57) ABSTRACT

Systems and methods of forming electrospun structures including pharmaceuticals are disclosed. An electrospun structure can include one or more polymers electrospun from a polymer solution comprising a pharmaceutical such that the pharmaceutical is dispersed throughout the one or more electrospun polymers. The pharmaceutical can include a cannabinoid, a phytocannabinoid, a terpene produced by a Cannabis plant, or a combination thereof. The one or more electrospun polymer are configured to degrade over a predetermined time period to deliver the pharmaceutical over the predetermined time period.

### CROSS-REFERENCE TO RELATED APPLICATIONS

This application claims priority to U.S. Provisional Application Ser. No. 62/979,818, filed Feb. 21, 2020, entitled ELECTROSPUN FIBERS HAVING A PHARMACEUTICAL AND METHODS OF MAKING AND USING THE SAME, which is hereby incorporated by reference herein in its entirety.

### BACKGROUND

Polymer fibers and other structures may be useful for the delivery of pharmaceuticals for treating injuries, illnesses, or disorders. Electrospinning is one method of fabricating such structures while finely controlling their properties and the amount of substance they include. In some instances, it may be useful for these structures to be used in place of other methods of pharmaceutical administration. Electrospun structures that include pharmaceuticals may be advantageous in these instances. In particular, there exists a need for electrospun polymer structures with one or more pharmaceuticals dispersed therein, which allows the structures to provide controlled delivery of the pharmaceuticals, whether implanted or administered orally, topically, or via other delivery mediums. In some embodiments, the pharmaceuticals dispersed in and/or administered via the electrospun polymer structures could include cannabinoids, phytocannabinoids (e.g., tetrahydrocannabinol or cannabidiol), terpenes produced by the Cannabis plant, and combinations thereof.

### SUMMARY

The present disclosure is directed to electrospun structures including pharmaceuticals, such as cannabinoids, phytocannabinoids (e.g., tetrahydrocannabinol or cannabidiol), terpenes produced by the Cannabis plant, and combinations thereof.

In some embodiments, there is provided an electrospun structure comprising: one or more polymers electrospun from a polymer solution comprising a pharmaceutical such that the pharmaceutical is dispersed throughout the one or more electrospun polymers; wherein the pharmaceutical comprises at least one of a cannabinoid, a phytocannabinoid, a terpene produced by a Cannabis plant, or a combination thereof; wherein the one or more electrospun polymers are configured to degrade over a predetermined time period to deliver the pharmaceutical over the predetermined time period.

In some embodiments, there is provided a method of fabricating an electrospun structure, the method comprising: dispersing a pharmaceutical into a polymer solution comprising one or more polymers; electrospinning the polymer solution to form the electrospun structure comprising the pharmaceutical dispersed throughout the one or more electrospun polymers of the electrospun structure; wherein the pharmaceutical comprises at least one of a cannabinoid, a phytocannabinoid, a terpene produced by a Cannabis plant, or a combination thereof; wherein the one or more electrospun polymers are configured to degrade over a predetermined time period to deliver the pharmaceutical over the predetermined time period.

In some embodiments, the pharmaceutical is present in an amount of at least 100 wt % relative to the one or more polymers.

In some embodiments, the one or more polymers comprise a first polymer and a second polymer.

In some embodiments, the first polymer and the second polymer were co-electrospun.

In some embodiments, the first polymer is configured to degrade over a first time period to release the pharmaceutical over the first time period and the second polymer is configured to degrade over a second time period to release the pharmaceutical over the second time period.

In some embodiments, the first time period and the second time period do not overlap.

In some embodiments, the first time period and the second time period at least partially overlap.

In some embodiments, the first polymer and the second polymer are arranged in a coaxial configuration.

In some embodiments, the one or more polymers are selected from the group consisting of poly(ethylene oxide), polyvinyl pyrrolidone, Dextran, saccharide, cellulose, chitosan, gelatin, collagen, polyvinyl alcohol, Eudragit, polyethylene terephthalate, polyester, polymethylmethacrylate, polyacrylonitrile, silicone, polyurethane, polycarbonate, polyether ketone ketone, polyether ether ketone, polyether imide, polyamide, polystyrene, polyether sulfone, polysulfone, polycaprolactone, polylactic acid, polylactide-co-caprolactone, polylactide-co-glycolide, polyglycolic acid, polyglycerol sebacic, polydiol citrate, polyhydroxy butyrate, polyether amide, polydioxanone, derivatives thereof, and combinations thereof.

In some embodiments, the electrospun structure is in the form of a sheet.

In some embodiments, the sheet has an average length of about 1 cm to about 6 cm, an average width of about 5 mm to about 10 mm, and an average thickness of about 1 mm to about 2 mm.

### BRIEF DESCRIPTION OF THE DRAWINGS

FIG. 1 is a scanning electron microscope (SEM) image of a fiber comprising a polymer and 50 wt % of a pharmaceutical based on the weight of the polymer, in accordance with an embodiment of the present disclosure.

FIG. 2 is a SEM image of a fiber comprising a polymer and 75 wt % of a pharmaceutical based on the weight of the polymer, in accordance with an embodiment of the present disclosure.

FIG. 3 is a graph comparing the plasma concentration of cannabidiol in a single pig when administered sublingually using electrospun structures as described herein (“strip”) and orally (“gummy”).

FIG. 4 is a graph comparing the plasma concentration of sildenafil citrate in a single pig when administered sublingually using electrospun structures as described herein (“strip”) and orally (“pill”).

FIG. 5 is a SEM image of an electrospun structure including a pharmaceutical, in accordance with an embodiment of the present disclosure.

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “pharmaceutical” is a reference to one or more pharmaceuticals and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

“Administering” when used in conjunction with a therapeutic means to administer a therapeutic directly into or onto a target tissue or to administer a therapeutic to a patient whereby the therapeutic positively impacts the tissue to which it is targeted. “Administering” a composition may be accomplished by injection, topical administration, oral administration, buccal administration, sublingual administration, transdermal administration, implantation, or by any of these methods in combination with other known techniques.

The term “subject” as used herein includes, but is not limited to, humans and non-human vertebrates such as wild, domestic, and farm animals.

By “pharmaceutically acceptable,” it is meant that the carrier, diluent or excipient must be compatible with the other ingredients of the formulation and not deleterious to the recipient thereof.

As used herein, the term “therapeutic” means an agent utilized to treat, combat, inhibit, ameliorate, prevent or improve an unwanted condition or disease of a patient. In part, embodiments herein are directed to the treatment of disorders or conditions. Disorders or conditions may include, in some non-limiting examples, seizure disorders such as epilepsy, pain, depression, mood disorders, and the like. Disorders or conditions may also include, for example, asthma, addictions, multiple sclerosis, immune disorders, allergies, anaphylaxis, chronic diseases, migraines, diabetes, cancer, and the like.

A “therapeutically effective amount” or “effective amount” of a composition is a predetermined amount calculated to achieve the desired effect, i.e., to ease, inhibit, block, or reverse a disorder. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages per day will normally fall within the range of from about 0.001 mg/kg to about 30 mg/kg, more usually in the range of from about 10 mg/kg to 20 mg/kg. Such dosages may be delivered once daily, once weekly, multiple times daily, or multiple times weekly. However, it will be understood that the effective amount administered will be determined by the physician in the light of the relevant circumstances including the condition to be treated, the choice of compound to be administered, and the chosen route of administration, and therefore the above dosage ranges are not intended to limit the scope of embodiments herein in any way. A therapeutically effective amount of compound of this invention is typically an amount such that when it is administered in a physiologically tolerable excipient composition, it is sufficient to achieve an effective systemic concentration or local concentration in the tissue.

The terms “treat,” “treated,” or “treating” as used herein refers to both therapeutic treatment and prophylactic or preventative measures, wherein the object is to inhibit, prevent or slow down (lessen) an undesired physiological condition, disorder or disease, or to improve, inhibit, or otherwise obtain beneficial or desired clinical results. For the purposes of this invention, beneficial or desired clinical results include, but are not limited to, improvement or alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

A number of pharmaceuticals can be used with the novel drug delivery mechanisms disclosed herein. In particular, a number of drug candidates have recently been developed that have high lipophilicity, high molecular weight, and poor water solubility. The poor water solubility of such drug candidates can lead to the failure of that drug candidate in development, or can lead to poor bioavailability, resulting in suboptimal drug delivery and even reduced patient compliance. A large percentage of drugs approved or in development have poor water solubility, and there exists a need for drug delivery mechanisms capable of improving their bioavailability.

For example, a select few constituents of the Cannabis plant have recently been on the forefront of emerging medical treatment for various diseases and symptoms. Cannabidiol (CBD), the second most prevalent phytocannabinoid in Cannabis, has been heavily researched due to its promising medicinal effects when administered to patients. CBD has been shown to have a wide range of beneficial properties, including being anti-inflammatory, anti-oxidant, anti-apoptotic, immune-modulatory, anti-epileptic, anti-anxiety, and more. However, unlike tetrahydrocannabinol (THC), the most prevalent and most commonly known phytocannabinoid in Cannabis, CBD does not produce psychoactive side effects. These widespread potential medicinal applications, combined with the lack of psychoactivity, make CBD an attractive option for the safe and effective use of Cannabis as a medical treatment. Cannabinoids can be selectively extracted from the Cannabis plant, purified, and dissolved in oil, which can be used in various ways to deliver the cannabinoid to a subject. Currently, CBD oil is typically delivered to a subject orally. The subject typically drinks multiple large quantities each day of CBD oil mixed with a carrier, such as sesame oil, to aid metabolization of the CBD oil. Subjects tend to find this delivery mode extremely undesirable for a number of reasons, including gastrointestinal side effects, taste, frequency, convenience, and efficacy in bioabsorportion of the drug. Additionally, some subjects may have difficulty swallowing due to their physical or mental impairments. Therefore, there exists a need for a simple delivery method that allows for the controlled release of high doses of CBD oil that result in a higher bioavailability of the compound while producing fewer unwanted side effects, thereby increasing a subject's compliance with a dosing regimen. Accordingly, in some embodiments, the pharmaceuticals used with the novel drug delivery mechanisms disclosed herein could include cannabinoids, phytocannabinoids (e.g., THC or CBD), terpenes produced by the Cannabis plant, and combinations thereof.

Other pharmaceuticals may also be used with the novel drug delivery mechanisms disclosed herein. Such pharmaceuticals may include, for example, steroids, anti-inflammatories, non-steroidal anti-inflammatories, analgesics, statins, antibiotics, antivirals, antifungals, antimicrobials, immunosuppressants, immunomodulators, antiproliferatives, sedatives, vitamins, hormones, growth factors, vasodilators, vasoconstrictors, antihistamines, opioids, and the like. Such pharmaceuticals may also include, for example, acetaminophen, cannabidiol, tetrahydrocannabinol, lovastatin, atorvastatin, caffeine, nicotine, insulin, sildenafil citrate, and the like.

### Electrospinning

Electrospinning is a method which may be used to process a polymer solution into a structure, such as a fiber. In embodiments wherein the diameter of the resulting fiber is on the nanometer scale, the fiber may be referred to as a nanofiber. Fibers may be formed into a variety of shapes by using a range of receiving surfaces, such as mandrels, molds, or collectors. The resulting fiber molds or shapes may be used in many applications, including the repair or replacement of biological structures. In some embodiments, the resulting structure (e.g., a fiber or fiber scaffold) may be implanted into a biological organism or a portion thereof.

Electrospinning methods may involve spinning a structure (e.g., a fiber) from a polymer solution by applying a high DC voltage potential between a polymer injection system and a receiving surface. In some embodiments, one or more charges may be applied to one or more components of an electrospinning system. In some embodiments, a charge may be applied to the receiving surface, the polymer injection system, the polymer solution, or combinations or portions thereof. Without wishing to be bound by theory, as the polymer solution is ejected from the polymer injection system, it is thought to be destabilized due to its exposure to a charge. The destabilized solution may then be attracted to a charged receiving surface. As the destabilized solution moves from the polymer injection system to the receiving surface, its solvents may evaporate and the polymer may stretch, leaving a long, thin fiber that is deposited onto the receiving surface. The polymer solution may form a Taylor cone as it is ejected from the polymer injection system and exposed to a charge. Further, polymers can be electrospun in a variety of different structures, including fibers, fibrous scaffolds, strips, patches, sheets, or shapes corresponding to anatomical structures. Still further, the structures can be electrospun using one or multiple polymers.

In some embodiments, multiple polymer types can be electrospun with each other to form structures in a process referred to as “co-electrospinning.” In co-electrospinning, two or more polymer solutions (containing the same or different polymer types) are ejected from different outlets and simultaneously electrospun with each other to form the resultant structure. Co-electrospinning creates two different fibers formed from the different polymer solutions that are intertwined with each other. The co-electrospun polymers can be spun from the same or different polymer solutions. The co-electrospun polymer types can have the same or different degradation rates. As one example, a first polymer type having a first degradation rate could be co-electrospun with a second polymer type having a second degradation rate, thereby creating an electrospun structure providing a time released profile that releases one or more pharmaceuticals contained within the different polymer types based on the differing degradation rates of the polymer types.

In some embodiments, multiple polymer types can be electrospun with each other to form structures in processes referred to as “coaxial electrospinning” or “multiaxial electrospinning.” In coaxial or multiaxial electrospinning, two or more polymer solutions (containing the same or different polymer types) are rejected from the same outlet and electrospun with each other to form the resultant structure. Coaxial or multiaxial electrospinning creates a single fiber composed of the different polymer types that has a core-shell structure. The coaxially or multi-axially electrospun polymer types can have the same or different degradation rates. As one example, a first polymer type having a first degradation rate could be coaxially or multi-axially electrospun with a second polymer type having a second degradation rate, thereby creating an electrospun structure providing a time released profile that releases one or more pharmaceuticals contained within the different polymer types based on the differing degradation rates of the polymer types.

In some embodiments, the co-electrospinning and coaxial or multiaxial electrospinning techniques described above could also be used in combination with each other. For example, coaxial polymers or fibers could be co-electrospun with each other. Accordingly, the various techniques described above can be used to electrospun structures having various timed release profiles for pharmaceuticals (which are described below) contained therein.

### Polymer Injection System

A polymer injection system may include any system configured to eject some amount of a polymer solution into an atmosphere to permit the flow of the polymer solution from the injection system to the receiving surface. In some embodiments, the polymer injection system may deliver a continuous or linear stream with a controlled volumetric flow rate of a polymer solution to be formed into a structure (e.g., a fiber). In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with one or more syringes containing one or more different polymer solutions. In some embodiments, the polymer injection system could include a rotating drum that dips into the polymer solution and ejects the solution as the drum rotates. In some embodiments, the polymer injection system could include a wire-based electrospinning system. In some embodiments, a charge may be applied to the polymer injection system, or to a portion thereof. In some embodiments, a charge may be applied to a needle or needle-like component of the polymer injection system. In one particular embodiment, the polymer injection system could include a wire electrode-based polymer injection system, such as the NS 8S1600U electrospinning production line available from ELMARCO®.

In some embodiments, the polymer solution may be ejected from the polymer injection system at a flow rate per needle of less than or equal to about 5 mL/h. Some non-limiting examples of flow rates per needle may include about 0.1 mL/h, about 0.5 mL/h, about 1 mL/h, about 1.5 mL/h, about 2 mL/h, about 2.5 mL/h, about 3 mL/h, about 3.5 mL/h, about 4 mL/h, about 4.5 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any ranges between any two of these values, including endpoints.

As the polymer solution travels from the polymer injection system toward the receiving surface, the diameter of the resulting fibers may be in the range of about 0.1 μm to about 10 μm. Some non-limiting examples of electrospun fiber diameters may include about 0.1 μm, about 0.2 μm, about 0.5 μm, about 1 μm, about 2 μm, about 5 μm, about 10 μm, about 15 μm, about 20 μm, or ranges between any two of these values, including endpoints.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include synthetic or semi-synthetic polymers such as, without limitation, poly(ethylene oxide), polyvinyl pyrrolidone, Dextran, saccharide, cellulose, chitosan, gelatin, collagen, polyvinyl alcohol, Eudragit, polyethylene terephthalate (PET), polyester, polymethylmethacrylate, polyacrylonitrile, silicone, polyurethane, polycarbonate, polyether ketone ketone, polyether ether ketone, polyether imide, polyamide, polystyrene, polyether sulfone, polysulfone, polyvinyl alcohol (PVA), polyvinyl acetate (PVAc), polycaprolactone (PCL), polylactic acid (PLA), polylactide co-caprolactone, polylactide co-glycolide, polyglycolic acid (PGA), polyglycerol sebacic, polydiol citrate, polyhydroxy butyrate, polyether amide, polydioxanone, copolymers thereof, and combinations or derivatives thereof. In some embodiments, the polymer solution may include a polymer that is a water-soluble polymer. Alternative polymer solutions used for electrospinning may include natural polymers such as fibronectin, collagen, gelatin, hyaluronic acid, chitosan, or combinations thereof. It may be understood that polymer solutions may also include a combination of synthetic polymers and naturally occurring polymers in any combination or compositional ratio. In some non-limiting examples, the polymer solution may comprise a weight percent ratio of, for example, poly(ethylene oxide) to polycaprolactone, from about 5% to about 90%. Non-limiting examples of such weight percent ratios may include about 5%, about 10%, about 15%, about 20%, about 25%, about 30%, about 33%, about 35%, about 40%, about 45%, about 50%, about 55%, about 60%, about 66%, about 70%, about 75%, about 80%, about 85%, about 90%, or ranges between any two of these values, including endpoints.

In some embodiments, the polymer may be present in an amount of about 1 wt % to about 30 wt % based on the weight of the polymer solution. In some non-limiting examples, the polymer may be present in the amount of, for example, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, or ranges between any two of these values, including endpoints.

In some embodiments, the polymer solution may comprise one or more solvents. In some embodiments, the solvent may comprise, for example, acetone, dimethylformamide, dimethylsulfoxide, N-methylpyrrolidone, N,N-dimethylformamide, acetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. Non-limiting examples of alcohols include ethanol, isopropanol, butanol, and the like. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

In some embodiments, the polymer solution may also include one or more pharmaceuticals. The pharmaceutical may comprise an oil, a liquid, a powder, a crystal, a droplet, or any other form known in the art. As noted herein, in some embodiments, the pharmaceutical can be incorporated into the polymer solution from which the structure is electrospun. Accordingly, the pharmaceutical is dispersed or otherwise incorporated throughout the entirety of the electrospun structure. These embodiments differ from pharmaceutical delivery vehicles (whether electrospun or not) that use a core/shell structure where the pharmaceutical is present only on the outside of the structure (i.e., in the shell) or only contained within the structure (i.e., within the core). In some embodiments, the pharmaceutical can be incorporated into the polymer solution in an amorphous state. In other embodiments, the pharmaceutical can be incorporated into the polymer solution in a crystalline state.

In some embodiments, the pharmaceutical may comprise an oil such as, for example, Cannabis oil, cannabinoid oil, CBD oil, olive oil, sesame oil, canola oil, palm oil, vegetable oil, castor oil, coconut oil, corn oil, soybean oil, derivatives thereof, or combinations thereof. In some embodiments, the pharmaceutical may comprise cannabinoids; phytocannabinoids, such as THC or pure CBD; terpenes produced by the Cannabis plant; and any combination thereof. In some embodiments, the pharmaceutical may also comprise one or more active ingredients. The active ingredients may include, for example, steroids, anti-inflammatories, non-steroidal anti-inflammatories, analgesics, statins, antibiotics, antivirals, antifungals, immunosuppressants, immunomodulators, antiproliferatives, sedatives, vitamins, hormones, growth factors, vasodilators, vasoconstrictors, antihistamines, opioids, derivatives thereof, or combinations thereof. In other examples, the active ingredients may include acetaminophen, CBD, tetrahydrocannabinol, lovastatin, atorvastatin, caffeine, nicotine, insulin, derivatives thereof, or combinations thereof.

In some embodiments, the pharmaceutical may comprise amiodarone, amlodipine, apixaban, atenolol, atorvastatin, benazepril, bisoprolol, candesartan, carvedilol, chlorthalidone, clonidine, clopidogrel, colesevelam, dabigatran, digoxin, diltiazem, dipyridamole, doxazosin, edoxaban, enalapril, enoxaparin, ezetimibe, felodipine, fenofibrate, fosinopril, furosemide, gemfibrozil, hydralazine, hydrochlorothiazide, irbesartan, isosorbide mononitrate, labetalol, lisinopril, losartan, lovastatin, metoprolol, nebivolol, niacin, nifedipine, nitroglycerin, olmesartan, omega-3-ethyl esters, prasugrel, pravastatin, propranolol, quinapril, ramipril, ranolazine, rivaroxaban, rosuvastatin, sacubitril/valsartan, simvastatin, spironolactone, telmisartan, terazosin, ticagrelor, tolvaptan, torsemide, triamterene/hydrochlorothiazide, valsartan, verapamil, warfarin, alendronate, canagliflozin, conjugated estrogen, cyanocobalamin, dapagliflozin, dexamethasone, dulaglutide, estradiol, ethinyl estradiol/etonogestrel, exenatide, folic acid, glimepiride, glipizide, ibandronate, levothyroxine, linagliptin, liraglutide, medroxyprogesterone, metformin, methylprednisolone, pioglitazone, prednisolone, prednisone, progesterone, raloxifene, repaglinide, risedronate, rosiglitazone, saxagliptin, sitagliptin, thyroid, dexlansoprazole, dicyclomine, diphenoxylate/atropine, esomeprazole, famotidine, lansoprazole, lubiprostone, metoclopramide, omeprazole, ondansetron, pantoprazole, polyethylene glycol, prochlorperazine, promethazine, rabeprazole, ranitidine, rifamixin, sucralfate, tizanidine, acyclovir, albendazole, amoxicillin, amoxicillin/clavulanate, atazanavir, azithromycin, cefdinir, cefuroxime, cephalexin, chlorhexidine, ciprofloxacin, clarithromycin, clindamycin, doxycycline, efavirenz emtricitabine/tenofovir disoproxil, entecavir, fidaxomicin, fluconazole, gatifloxacin, hydroxychloroquine, imiquimod, ketoconazole, levofloxacin, maraviroc, metronidazole, minocycline, moxifloxacin, mupirocin, nitazoxanide, nitrofurantoin, nystatin, oseltamivir, penicillin, phenazopyridine, raltegravir, sofosbuvir, terbinafine, trimethoprim/sulfamethoxazole, valacyclovir, allopurinol, colchicine, epinephrine, etanercept, tacrolimus, adapalene, bimatoprost, brimonidine clobetasol, clotrimazole/betamethasone, cyclosporine, darifenacin, dutasteride, febuxostat, finasteride, fluocinonide, hydrocortisone, latanoprost, loteprednol, oxybutynin, prenatal 1+1, sildenafil, solifenacin, tadalafil, tamsulosin, testosterone, tolterodine, travoprost, triamcinolone, vardenafil, potassium chloride, potassium iodide, alprazolam, amitriptyline, aripiprazole, atomoxetine, baclofen, benztropine, bupropion, buspirone, carbamazepine, carbidopa/levodopa, citalopram, clobazam, clonazepam, desvenlafaxine, dexmethylphenidate, diazepam, divalproex, donepezil, doxepin, duloxetine, eletriptan, escitalopram, eszopiclone, fluoxetine, gabapentin, guanfacine, lacosamide, lamotrigine, levetiracetam, lisdexamfetamine, lithium, lorazepam, lurasidone, meclizine, memantine, metaxalone, methylphenidate, mirtazapine, modafinil, nortriptyline, olanzapine, oxcarbazepine, paroxetine, phenobarbital, phentermine, phenytoin, pramipexole, pregabalin, quetiapine, risperidone, rizatriptan, ropinirole, sertraline, sumatriptan, temazepam, topiramate, trazodone, venlafaxine, vilazodone, ziprasidone, zolpidem, azathioprine, anastrozole, darbepoetin, epoetin alfa, methotrexate, tamoxifen, buprenorphine/naloxone, carisoprodol, celecoxib, tylenol (with and without codeine), cyclobenzaprine, diclofenac sodium, etodolac, fentanyl, hydrocodone (with and without acetaminophen), ibuprofen, indomethacin, lidocaine, meloxicam, methadone, methocarbamol, morphine, nabumetone, naproxen, oxycodone, pentosan, tramadol, albuterol, azelastine, benzonatate, budesonide, budesonide/formoterol, cetirizine, fexofenadine, fluticasone, salmeterol, hydroxyzine, ipratropium/albuterol, levalbuterol, levocetirizine, mometasone, montelukast, olopatadine, tiotropium, triamcinolone, varenicline, or any combination thereof.

In some embodiments, the pharmaceutical may be dispersed in a solution different from the polymer solution described in other embodiments herein. In some embodiments, the pharmaceutical may be dispersed in the polymer solution. In other embodiments, the pharmaceutical can be dispersed in a separate solution prior to being added to the polymer solution. In some embodiments, the pharmaceutical may comprise a crystal. The crystal may have a first dimension from about 10 nm to about 10 μm. In certain embodiments, the first dimension may describe the length, width, or height of the crystal, or a combination thereof. In other embodiments, the pharmaceutical may comprise a crystal dissolved in an oil.

The type of polymer in the polymer solution may determine the characteristics of the electrospun structure. Some structures may be composed of polymers that are bio-stable and not absorbable or biodegradable when implanted. Such structures may remain generally chemically unchanged for the length of time in which they remain implanted. Alternatively, structures may be composed of polymers that may be absorbed or biodegraded over time. Such structures may act as an initial template or scaffold for the repair or replacement of organs and/or tissues. These organ or tissue templates or scaffolds may degrade in vivo once the tissues or organs have been replaced or repaired by natural structures and cells. Alternatively, such structures may degrade or disintegrate at a faster controlled rate, such as a rate appropriate for drug delivery rather than cell or tissue ingrowth. It may be further understood that a polymer solution and its resulting electrospun structure(s) may be composed of more than one type of polymer, and that each polymer therein may have a specific characteristic, such as bio-stability or biodegradability.

In some embodiments, the pharmaceutical can be prepared, incorporated into the polymer solution, or otherwise processed at various temperatures. In one embodiment, the pharmaceutical can be processed at room or ambient temperature. For example, the pharmaceutical could be processed at 20 to 25° C.

In some embodiments, the pharmaceutical can be provided in a variety of different dosages that vary based on the type of pharmaceutical and the intended use of the pharmaceutical-bearing electrospun structure. For example, the pharmaceuticals in the electrospun structures described herein could be provided in different dosages depending upon the ability of the subject to receive or ingest the electrospun structures. In particular, some individuals or animals may have difficulty swallowing (e.g., due to a medical condition or, in the case of animals, an unwillingness to swallow the electrospun structures). Accordingly, in some embodiments, the dosage of the pharmaceutical in the electrospun structures could be increased to minimize the amount of electrospun structures that are required to be delivered to and swallowed by the person or animal.

### Applying Charges to Electrospinning Components

In an electrospinning system, one or more charges may be applied to one or more components, or portions of components, such as, for example, a receiving surface, a polymer injection system, a polymer solution, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to the polymer solution, or portions thereof. In some embodiments, a negative charge may be applied to the polymer solution, or portions thereof. In some embodiments, the polymer solution, or portions thereof, may be grounded. In some embodiments, a positive charge may be applied to the receiving surface, or portions thereof. In some embodiments, a negative charge may be applied to the receiving surface, or portions thereof. In some embodiments, the receiving surface, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about −100 kV to about 100 kV, including endpoints. In some non-limiting examples, the charge applied to any component of the electrospinning system, or portions thereof, may be about −100 kV, about −75 kV, about −50 kV, about −30 kV, about −25 kV, about −15 kV, about −10 kV, about −5 kV, about −3 kV, about −1 kV, about −0.01 kV, about 0.01 kV, about 1 kV, about 5 kV, about 10 kV, about 12 kV, about 15 kV, about 20 kV, about 25 kV, about 30 kV, about 50 kV, about 75 kV, about 100 kV, or any range between any two of these values, including endpoints. In some embodiments, any component of the electrospinning system, or portions thereof, may be grounded.

### Receiving Surface Movement During Electrospinning

During electro spinning, in some embodiments, the receiving surface may move with respect to the polymer injection system. In some embodiments, the polymer injection system may move with respect to the receiving surface. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the receiving surface may be in contact with or mounted upon a support structure that may be moved using one or more motors or motion control systems. The pattern of the electrospun structure deposited on the receiving surface may depend upon the one or more motions of the receiving surface with respect to the polymer injection system. In some embodiments, the receiving surface may be configured to rotate about its long axis. In one non-limiting example, a receiving surface having a rotation rate about its long axis that is faster than a translation rate along a linear axis may result in a nearly helical deposition of an electrospun fiber, forming windings about the receiving surface. In another example, a receiving surface having a translation rate along a linear axis that is faster than a rotation rate about a rotational axis may result in a roughly linear deposition of an electrospun fiber along a linear extent of the receiving surface. In some embodiments, the electrospinning system could include a roller electrospinning system.

### Electrospun Structures Having Pharmaceuticals

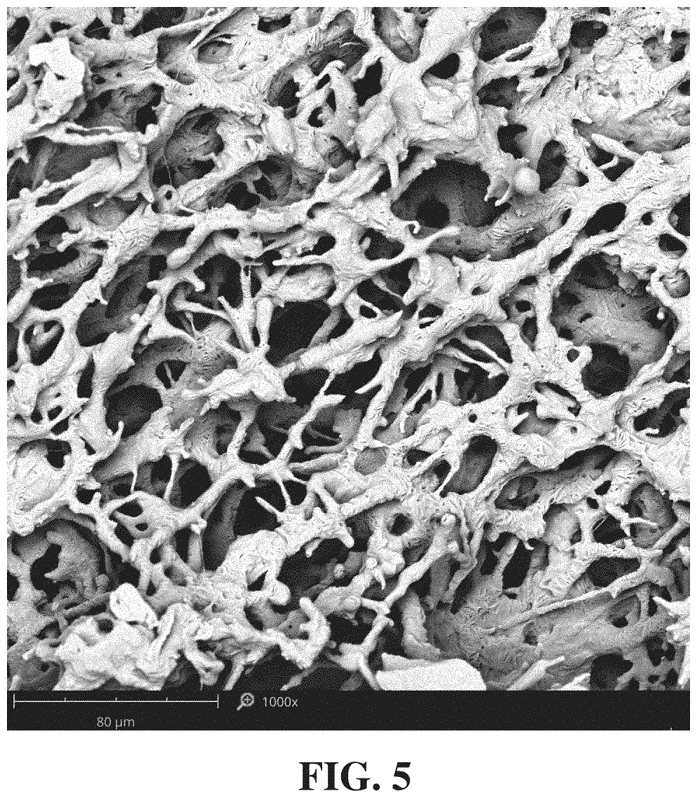

In some embodiments, a structure may be electrospun from a polymer solution including one or more pharmaceuticals using any of the electrospinning techniques and electrospinning systems described above. In embodiments where the pharmaceutical is dispersed within the polymer solution, the structure electrospun from the polymer solution thus includes the pharmaceutical dispersed throughout. In certain embodiments, the pharmaceutical is dispersed throughout the electrospun polymer structure, rather than the pharmaceutical being present only on the outer surface of the electrospun structure as with pharmaceutical delivery vehicles that use a core/shell structure. Such embodiments exclude dipping, spraying, or otherwise treating the outside surface of an electrospun structure with pharmaceuticals. One benefit of these embodiments is that the pharmaceuticals being dispersed within and throughout the electrospun polymer structure makes the electrospun structure resistant to accidental or unanticipated removal of the pharmaceutical therefrom. An SEM image of an illustrative embodiment of an electrospun structure including a pharmaceutical dispersed therein is shown in FIG. 5.

In some embodiments, the electrospun polymer may comprise one or more polymers. In some embodiments, the polymers may include, without limitation, the polymers described above. In some embodiments, the polymer may comprise a water-soluble polymer. It may be understood that polymers may also include a combination of synthetic polymers and naturally occurring polymers in any combination or compositional ratio.

In some embodiments, the pharmaceutical may comprise an oil. In certain embodiments, oil may comprise, for example, Cannabis oil, cannabinoid oil, CBD oil, olive oil, sesame oil, canola oil, palm oil, vegetable oil, derivatives thereof, or combinations thereof. In some embodiments, the pharmaceutical may comprise a crystal. In some embodiments, the pharmaceutical may comprise an oil dispersed or dissolved in a solution. In other embodiments, pharmaceutical may comprise a crystal dispersed or dissolved in a solution. In certain embodiments, the pharmaceutical may comprise a crystal dispersed or dissolved in an oil. In an embodiment, the pharmaceutical may comprise a CBD crystal dispersed or dissolved in an oil.

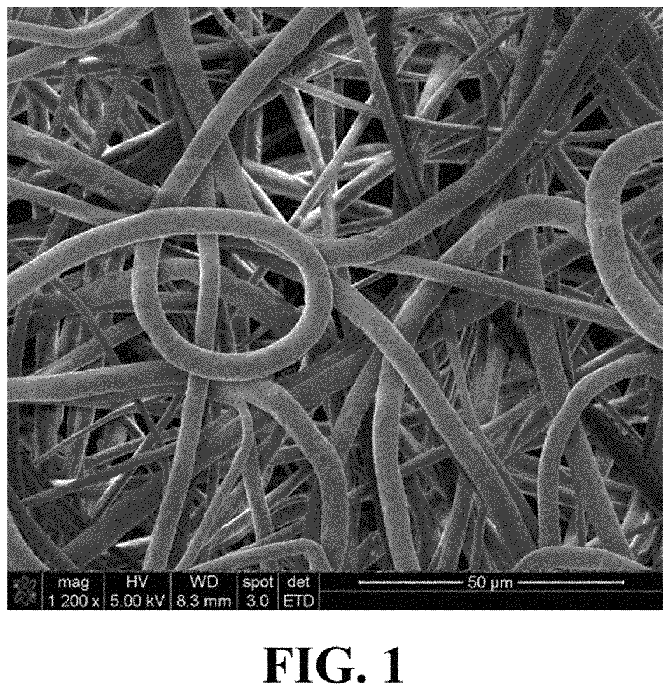

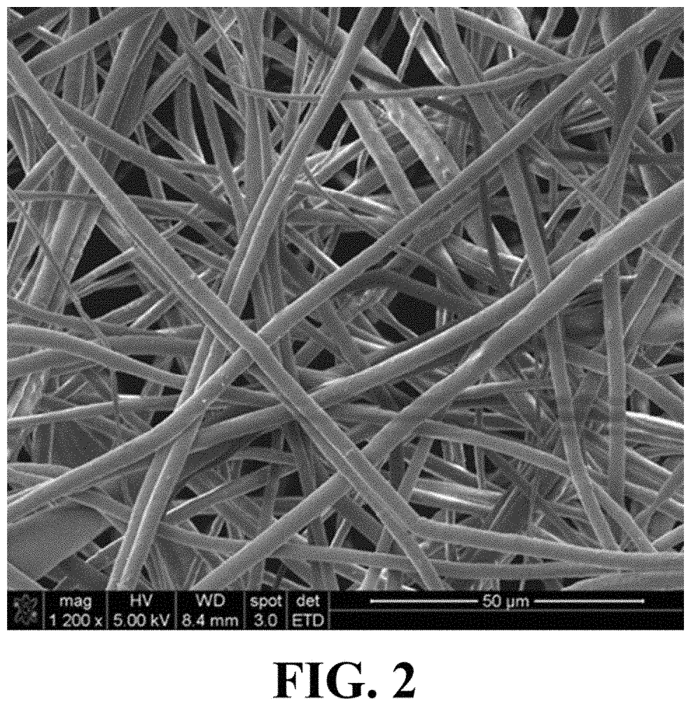

In some embodiments, the pharmaceutical may be present in an amount of about 1 wt % to about 1,500 wt %, based on the weight of the polymer. The term “wt %” as used herein refers to the percent weight of the identified material based on the total weight of a formulation containing the identified material. For example, a pharmaceutical being present in an amount of about 500 wt %, based on the weight of a polymer equates to a final formulation where the concentration of the pharmaceutical is five times greater than the total weight of the polymer. In one embodiment, the pharmaceutical may be present in an amount of about 1 wt % to about 1,500 wt %. In some embodiments, pharmaceutical may be present in an amount of about 50 wt % to about 75 wt %. In some embodiments, the pharmaceutical may be present in an amount of, for example, about 1 wt %, about 2 wt %, about 3 wt %, about 4 wt %, about 5 wt %, about 6 wt %, about 7 wt %, about 8 wt %, about 9 wt %, about 10 wt %, about 20 wt %, about 30 wt %, about 40 wt %, about 50 wt %, about 60 wt %, about 70 wt %, about 80 wt %, about 90 wt %, about 100 wt %, about 110 wt %, about 120 wt %, about 130 wt %, about 140 wt %, about 150 wt %, about 160 wt %, about 170 wt %, about 180 wt %, about 190 wt %, about 200 wt %, about 210 wt %, about 220 wt %, about 230 wt %, about 240 wt %, about 250 wt %, about 260 wt %, about 270 wt %, about 280 wt %, about 290 wt %, about 300 wt %, about 310 wt %, about 320 wt %, about 330 wt %, about 340 wt %, about 350 wt %, about 360 wt %, about 370 wt %, about 380 wt %, about 390 wt %, about 400 wt %, about 410 wt %, about 420 wt %, about 430 wt %, about 440 wt %, about 450 wt %, about 460 wt %, about 470 wt %, about 480 wt %, about 490 wt %, about 500 wt %, about 510 wt %, about 520 wt %, about 530 wt %, about 540 wt %, about 550 wt %, about 560 wt %, about 570 wt %, about 580 wt %, about 590 wt %, about 600 wt %, about 610 wt %, about 620 wt %, about 630 wt %, about 640 wt %, about 650 wt %, about 660 wt %, about 670 wt %, about 680 wt %, about 690 wt %, about 700 wt %, about 710 wt %, about 720 wt %, about 730 wt %, about 740 wt %, about 750 wt %, about 760 wt %, about 770 wt %, about 780 wt %, about 790 wt %, about 800 wt %, about 810 wt %, about 820 wt %, about 830 wt %, about 840 wt %, about 850 wt %, about 860 wt %, about 870 wt %, about 880 wt %, about 890 wt %, about 900 wt %, about 910 wt %, about 920 wt %, about 930 wt %, about 940 wt %, about 950 wt %, about 960 wt %, about 970 wt %, about 980 wt %, about 990 wt %, about 1,000 wt %, about 1,110 wt %, about 1,120 wt %, about 1,130 wt %, about 1,140 wt %, about 1,150 wt %, about 1,160 wt %, about 1,170 wt %, about 1,180 wt %, about 1,190 wt %, about 1,200 wt %, about 1,210 wt %, about 1,220 wt %, about 1,230 wt %, about 1,240 wt %, about 1,250 wt %, about 1,260 wt %, about 1,270 wt %, about 1,280 wt %, about 1,290 wt %, about 1,300 wt %, about 1,310 wt %, about 1,320 wt %, about 1,330 wt %, about 1,340 wt %, about 1,350 wt %, about 1,360 wt %, about 1,370 wt %, about 1,380 wt %, about 1,390 wt %, about 1,400 wt %, about 1,410 wt %, about 1,420 wt %, about 1,430 wt %, about 1,440 wt %, about 1,450 wt %, about 1,460 wt %, about 1,470 wt %, about 1,480 wt %, about 1,490 wt %, about 1,500 wt %, or ranges between any two of these values, including endpoints, based on the weight of the polymer. For example, FIG. 1 and FIG. 2 show scanning electron microscope (SEM) images of electrospun polymer fibers comprising 50 wt % and 75 wt %, respectively, of a pharmaceutical based on the weight of the polymer.

Another benefit of the embodiments disclosed herein is that electrospinning polymers including pharmaceuticals provides a mechanism to include high concentrations of a pharmaceutical within a structure electrospun from the polymers. Other processing methods, such as extrusion or coating techniques, are limited in the amount of pharmaceutical that can be present within the extruded polymer. In particular, because extruded polymers require some degree of mechanical integrity in order to withstand the extruding process, extruded polymers cannot hold high concentrations of pharmaceuticals. An extruded polymer having a high pharmaceutical content will exhibit an increase in viscosity and/or will result in a final extruded product having no, or poor, mechanical integrity. In contrast, the electrospun structures disclosed herein are capable of being formed from electrospun polymers having a high pharmaceutical content, while at the same time providing an electrospun structure having a high degree of mechanical integrity. Electrospun polymers, as described herein, may be loaded with a high concentration of a pharmaceutical. Examples of such high loading concentrations are disclosed herein. The high loading concentrations of the electrospun polymers unexpectedly result in a structure that maintains sufficient tensile strength, modulus, and elongation as compared to a structure produced via a typical melt process, i.e., extruding, which suffers extreme loss of strength and elongation. For example, in one embodiment, an electrospun polymer described herein can be loaded with about 100 wt % of a pharmaceutical, resulting in a structure that maintains sufficient tensile strength, modulus, and elongation. Typical melt-processing techniques are limited in the amount of pharmaceutical that can be present in a polymer before suffering losses in mechanical integrity in an extruded product. For example, increases in filler content of a typical polyvinyl chloride (PVC) formulation have been shown to decrease the extension at break and tensile strength. Further, thermoset systems also illustrate a decrease in tensile strength and elongation (%) with increasing filler content. Typical melt process systems have been shown to have the best synthetic properties at about a 25% filler load, after which the synthetic properties vastly erode. Thus, filler loadings of typical melt process polymers of over around 25%-50 wt % have been shown to negatively affect strength, elongation and other mechanical properties of typical melt-process polymeric systems. In contrast, it has been observed that fibers according to an embodiment of the instant disclosure, i.e., fibers made from electrospun polymers having high concentrations of filler (including pharmaceuticals), retain sufficient mechanical integrity that is unexpected in view of the prior art melt-process systems. Furthermore, the electrospinning process may be performed at room temperature, a safe processing temperature for pharmaceuticals that generally will not disrupt their structure, purity, or integrity. Processing at room temperature makes the methods disclosed herein more desirable than heated processes, such as melt extrusion, that may damage pharmaceuticals. In addition, an electrospun polymer having a high concentration of a pharmaceutical results in a structure capable of delivering an effective amount of a pharmaceutical to a subject in a tolerable dosing manner, for example. Therefore, in some embodiments, it is desirable to maximize the concentration of the pharmaceutical in the electrospun polymers. In some embodiments, the pharmaceutical may be present in an amount that maximizes the concentration of the pharmaceutical in the electrospun polymer while at the same time retaining the integrity of a structure formed from the polymer solution.

In some embodiments, an electrospun fiber may have a length from about 5 μm to about 5 m. In some embodiments, the fiber may have a length of, for example, about 5 μm, about 10 μm, about 20 μm, about 30 μm, about 40 μm, about 50 μm, about 60 μm, about 70 μm, about 80 μm, about 90 μm, about 100 μm, about 150 μm, about 200 μm, about 250 μm, about 300 μm, about 400 μm, about 500 μm, about 600 μm, about 700 μm, about 800 μm, about 900 μm, about 1 mm, about 5 mm, about 10 mm, about 20 mm, about 30 mm, about 40 mm, about 50 mm, about 60 mm, about 70 mm, about 80 mm, about 90 mm, about 100 mm, about 150 mm, about 200 mm, about 250 mm, about 300 mm, about 350 mm, about 400 mm, about 450 mm, about 500 mm, about 550 mm, about 600 mm, about 650 mm, about 700 mm, about 750 mm, about 800 mm, about 850 mm, about 900 mm, about 950 mm, about 1 m, about 2 m, about 3 m, about 4 m, about 5 m, or ranges between any two of these values, including endpoints. In some embodiments, an electrospun structure can be fabricated from one or more electrospun fibers, electrospun fiber fragments, or combinations thereof.

In some embodiments, an electrospun fiber may have a diameter of about 50 nm to about 50 μm. In some embodiments, an electrospun fiber may have a diameter of, for example, about 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1 μm, about 2 μm, about 3 μm, about 4 μm, about 5 μm, about 10 μm, about 15 μm, about 20 μm, about 25 μm, about 30 μm, about 35 μm, about 40 μm, about 45 μm, about 50 μm, or ranges between any two of these values, including endpoints.

In some embodiments, the electrospun structure may be formed into a shape such as, for example, a fragment, a cluster, a strand, a thread, a rope, a braid, a sheet, a coil, a tube, a cylinder, a textile, or a mold of an organ. In some embodiments, the structure may be formed into a mold of an organ such as, for example, a trachea, a trachea and at least a portion of at least one bronchus, a trachea and at least a portion of a larynx, a larynx, an esophagus, a large intestine, a small intestine, an upper bowel, a lower bowel, a vascular structure, an artery, a vein, a nerve conduit, a ligament, a tendon, and portions thereof. In some embodiments, the structure may be formed into the shape of a suture.

In some embodiments, the electrospun structure could include a sheet, strip, or patch. In some embodiments, the electrospun structures could have an average length of about 1 cm to about 6 cm, an average width of about 5 mm to 10 mm, and an average thickness of about 1 mm to about 2 mm. The average length of the sheet may be, for example, about 1 cm, about 2 cm, about 3 cm, about 4 cm, about 5 cm, about 6 cm, or any range between any two of these values, including endpoints. The average width of the sheet may be, for example, about 5 mm, about 6 mm, about 7 mm, about 8 mm, about 9 mm, about 10 mm, or any range between any two of these values, including endpoints. The average thickness of the sheet may be, for example, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, or any range between any two of these values, including endpoints.

In some embodiments, the electrospun structures disclosed herein can be configured to degrade in a particular manner or over a particular time period to provide a defined release profile for the pharmaceutical. In other words, when the electrospun structures are contacted with a subject's body (e.g., when ingested, applied topically, or applied sublingually), the polymers of the structure degrade (either naturally or via the mechanisms of the subject's body), which causes the pharmaceutical dispersed within the electrospun structure to be released to the subject. Accordingly, the electrospun structures can be configured to predictably control the timed release of the pharmaceutical. In some embodiments, the structures can be electrospun from a polymer type that degrades over a defined time period. In some embodiments, the structures can be electrospun from a combination or blend of polymer types that degrade over different or the same time periods. For example, the electrospun structure could comprise a first polymer material that degrades over a first time period and a second polymer material that degrades over a second time period. Accordingly, such an electrospun structure would release a first amount of the pharmaceutical (dispersed within the first polymer material) over the first time period and a second amount of the pharmaceutical (dispersed within the second polymer material) over the second time period. The time periods could overlap with each other or be non-overlapping. The different polymer materials could be electrospun with each other to form the electrospun structure using a variety of different techniques, including co-electrospinning, coaxial electrospinning, multiaxial electrospinning, or a combination thereof, for example. In some embodiments, the structures can be fabricated in a particular configuration to control the degradation of the structure and, thus, the release profile of the pharmaceutical. In one embodiment, the electrospun structure can comprise a core-shell electrospun structure that was coaxially electrospun using a first polymer solution and a second polymer solution that are arranged in a coaxial configuration. The first and second polymer solutions could be the same or different polymer materials. In another embodiment, the electrospun structure can comprise a core-shell electrospun structure that was multi-axially electrospun using three polymer solutions arranged in a tri-axial configuration. In yet another embodiment, the electrospun structure can comprise multiple electrospun polymers co-electrospun with each other, thereby forming multiple intertwined polymer fibers.

In certain embodiments, the electrospun structures described herein may be used to treat a number of disorders, including any disorders for which any pharmaceutical is known to be effective. Such disorders may include, for example, seizure disorders such as epilepsy, motor disorders such as Parkinson's disease and tremors, depression, anxiety, mood disorders, personality disorders, sleep disorders, traumatic brain injuries, Alzheimer's disease, neurodegenerative disorders, pain, and the like. Such disorders may also include, for example, asthma, addictions, multiple sclerosis, immune disorders, allergies, anaphylaxis, chronic diseases, migraines, diabetes, cancer, and the like.

In one embodiment, the electrospun structures described herein may be used to treat, for example, a seizure disorder in a subject. A method for treating a disorder in a subject may include obtaining a structure comprising an electro spun polymer and an effective amount of a pharmaceutical, applying the structure to a region of the subject, and allowing the electrospun structure to disintegrate. In such an embodiment, the electrospun structures may comprise, for example, poly(ethylene oxide) and CBD. In certain embodiments, the pharmaceutical may be present in an amount of about 100 wt % based on the weight of the electrospun polymer. In some embodiments, the disorder may be a seizure disorder. In other embodiments, the disorder may be any of the disorders described herein.

In various embodiments, the pharmaceutical-impregnated electrospun polymer structures described herein could be delivered in a variety of different manners and/or via a variety of different orifices or membranes. In particular, the pharmaceutical-impregnated electrospun polymer structures could be delivered via a variety of different administration techniques, including oral administration, topical administration, buccal administration, sublingual administration, vaginal administration, anal administration, ophthalmic administration, and/or nasal administration. The pharmaceuticals could be administered via any membrane or orifice that causes the electrospun polymer structures to disintegrate when in contact therewith. In these embodiments, the electrospun polymer structures could be applied to a region of an individual's body (e.g., the buccal region or the sublingual region) and allowed to disintegrate over a time period to deliver the pharmaceutical to the individual over the time period.

In another embodiment, a method may include obtaining a structure comprising an electrospun polymer and an effective amount of a pharmaceutical, implanting the structure within a subject, and allowing the structure to disintegrate. In certain embodiments, the pharmaceutical may be present in an amount of about 100 wt % based on the weight of the electrospun polymer.

As one example implementation, incorporating a pharmaceutical such as CBD oil with an electrospun polymer structure, as described herein, allows for the production of a fibrous scaffold containing the cannabidiol compound. If such a scaffold is made by selecting a polymer with a desirable degradation rate in vivo, then the resulting scaffold could be a promising candidate for drug delivery. A fiber scaffold comprising poly(ethylene oxide) and CBD oil, for example, could be used as an orally disintegrating tablet or scaffold for the delivery of CBD because of its ability to comprise about 100% oil without losing its mechanical characteristics. Changing the dimensions of the fibrous scaffold for use in the methods described herein and changing the frequency of administration may represent simple methods for altering drug dosage for a subject. When administered buccally, the fibrous scaffold may disintegrate in the subject's mouth as it is absorbed into the body, thereby bypassing the first-pass metabolism and eliminating gastrointestinal side effects associated with other delivery modes of CBD. When compared to the current method of delivering CBD oil to a subject, which typically involves the subject drinking a large quantity of CBD oil mixed with a carrier, such as sesame oil, multiple times each day, a scaffold comprising the fibers described herein may allow for a surprisingly improved bioavailability of the pharmaceutical being administered. The administration methods as described herein may also improve subject compliance with a particular dosing regimen, regardless of the subject's ability to swallow or take other oral medications. The administration methods described herein may also improve the precision with which a particular pharmaceutical dosage is included in a fiber, especially when compared to inexact combinations of drugs in carrier oils.

### EXAMPLES

### Example 1: Oil Dispersed Within Electrospun Polymer Fibers

In some examples, polymers (e.g., poly(ethylene oxide) and Eudragit's L-100) were used as water soluble and pH-sensitive polymer carriers for the oils. Polymer solutions containing poly(ethylene oxide) dissolved in dichloromethane were able to produce continuous electrospun fibers with loadings of the oil component as high as 1:1 with respect to the mass of the oil to the mass of the solid polymer in solution. The following oils were tested, using a 1:2 ratio of the mass of the oil to the mass of the solid polymer in solution, and produced continuous electrospun fibers: vegetable oil, canola oil, olive oil, and sesame oil. Solutions comprised of olive oil, hexafluoro isopropanol, and poly(glycolic acid) or poly(vinyl acetate) were electrospun, and produced continuous electrospun fibers. These solutions were formed into fibers via electrospinning. The oil-loaded polymer solutions were loaded into syringes and pumped through a 20 gage needle at a flow rate of 5 mL/hr while a positive voltage of 10 kV-13 kV was applied to the metal needle tip. This charged needle tip was positioned 20-30 cm away from a collection surface that had an applied voltage of −5 kV. The material produced in this manner was accumulated until a fiber mat of approximately 0.1 mm in thickness was produced. For example, FIGS. 1 and 2 are scanning electron microscope (SEM) images of electrospun polymer fibers comprising 50 wt % and 75 wt %, respectively, of a pharmaceutical (in an oil form) based on the weight of the polymer.

### Example 2: Oral Vs. Sublingual Delivery of Cannabidiol in a Porcine Model

Four healthy Yorkshire pigs were selected to receive both oral and sublingual administrations of cannabidiol (i.e., each animal received both an oral administration and a sublingual administration separated by 48 hours). For the sublingual administration, a scaffold comprising fibers as described herein was used for each animal. Specifically, the fibers were electrospun using a polymer solution comprising cannabidiol and 13 wt % 100,000 molecular weight polyethylene oxide dissolved in dichloromethane. The cannabidiol was dispersed within the resulting electrospun polyethylene oxide fibers, as described herein. The resulting electrospun polyethylene oxide fibers comprised 40 wt % cannabidiol (relative to the polyethylene oxide mass) and 50 wt % confectionary sugar (i.e., for every 10 mg of polyethylene oxide, 4 mg of cannabidiol and 5 mg of confectionary sugar were added).

The scaffold was placed under the tongue of each animal and allowed to dissolve in place. A catheter was placed in the animal's vein and used to withdraw blood at baseline and at 3, 9, 15, 30, 45, 60, and 120 minutes post-administration.

For the oral administration, each animal received a gummy (given orally) containing the same amount of cannabidiol contained within the scaffold used for the sublingual administration (a total of 10 mg of cannabidiol for each administration). The venous catheter was again used to withdraw blood at baseline and at 3, 9, 15, 30, 45, 60, and 120 minutes post-administration.

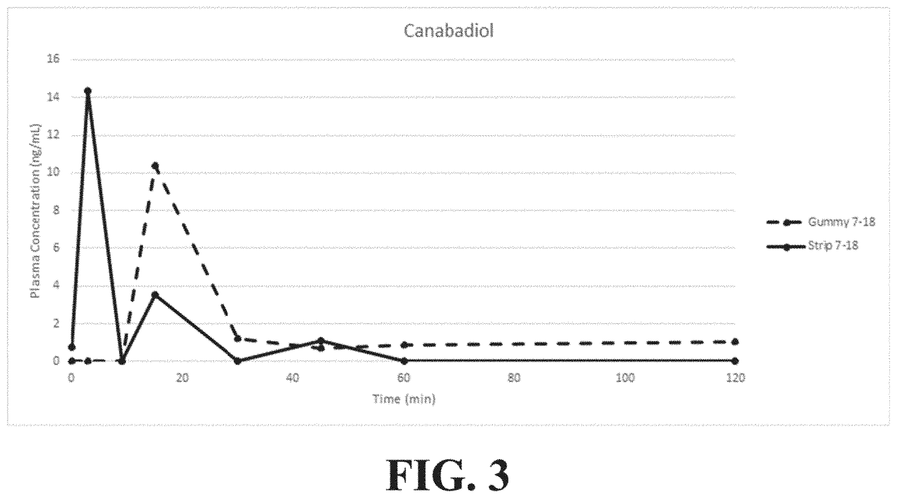

Each blood sample was analyzed using high-performance liquid chromatography (HPLC) to determine the plasma concentration of cannabidiol in ng/mL. FIG. 3 is a graph showing the plasma concentration of cannabidiol in a representative animal (number 7-18) when administered sublingually using fibers as described herein (“strip”) and orally (“gummy”). FIG. 3 shows that the sublingual administration reached a higher plasma concentration in a shorter period of time compared to the oral administration.

### Example 3: Oral Vs. Sublingual Delivery of Sildenafil Citrate in a Porcine Model

Four healthy Yorkshire pigs were selected to receive both oral and sublingual administrations of sildenafil citrate (i.e., each animal received both an oral administration and a sublingual administration separated by 48 hours). For the sublingual administration, a scaffold comprising fibers as described herein was used for each animal. Specifically, the fibers comprised were electrospun using a polymer solution comprising sildenafil citrate and 13 wt % 100,000 molecular weight polyethylene oxide dissolved in dichloromethane. The sildenafil citrate was dispersed within the resulting electrospun polyethylene oxide fibers, as described herein. The resulting electrospun polyethylene oxide fibers comprised 25 wt % sildenafil citrate (relative to the polyethylene oxide mass) and 50 wt % confectionary sugar (i.e., for every 10 mg of polyethylene oxide, 2.5 mg of sildenafil citrate and 5 mg of confectionary sugar were added).

The scaffold was placed under the tongue of each animal and allowed to dissolve in place. A catheter was placed in the animal's vein and used to withdraw blood at baseline and at 3, 9, 15, 30, 45, 60, and 120 minutes post-administration.

For the oral administration, each animal received a pill (given orally) containing the same amount of sildenafil citrate contained within the scaffold used for the sublingual administration (a total of 25 mg of sildenafil citrate for each administration). The venous catheter was again used to withdraw blood at baseline and at 3, 9, 15, 30, 45, 60, and 120 minutes post-administration.

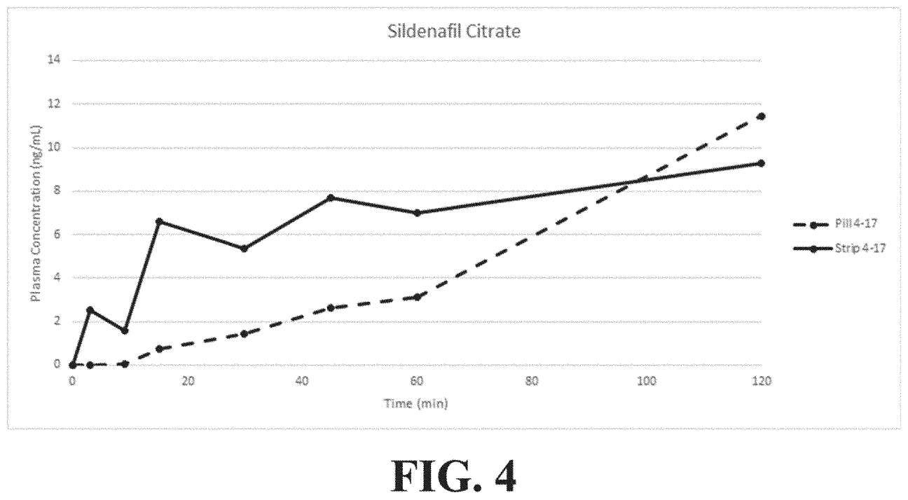

Each blood sample was analyzed using HPLC to determine the plasma concentration of sildenafil citrate in ng/mL. FIG. 4 is a graph showing the plasma concentration of sildenafil citrate in a representative animal (number 4-17) when administered sublingually using fibers as described herein (“strip”) and orally (“pill”). FIG. 4 shows that the sublingual administration reached a higher plasma concentration in a shorter period of time compared to the oral administration, and that the sublingual administration maintained the higher plasma concentration over a longer period of time.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1. An electrospun structure comprising:
one or more polymers electrospun from a polymer solution comprising a pharmaceutical such that the pharmaceutical is dispersed throughout the one or more electrospun polymers;
wherein the pharmaceutical comprises at least one of a cannabinoid, a phytocannabinoid, a terpene produced by a Cannabis plant, or a combination thereof;
wherein the one or more electrospun polymers are configured to degrade over a predetermined time period to deliver the pharmaceutical over the predetermined time period.

2. The electrospun structure of claim 1, wherein the pharmaceutical is present in an amount of at least 100 wt % relative to the one or more polymers.

3. The electrospun structure of claim 1, wherein the one or more polymers comprise a first polymer and a second polymer.

4. The electrospun structure of claim 3, wherein the first polymer and the second polymer were co-electrospun.

5. The electrospun structure of claim 3, wherein:
the first polymer is configured to degrade over a first time period to release the pharmaceutical over the first time period and the second polymer is configured to degrade over a second time period to release the pharmaceutical over the second time period.

6. The electrospun structure of claim 5, wherein the first time period and the second time period do not overlap.

7. The electrospun structure of claim 5, wherein the first time period and the second time period at least partially overlap.

8. The electrospun structure of claim 3, wherein the first polymer and the second polymer are arranged in a coaxial configuration.

9. The electrospun structure of claim 1, wherein the one or more polymers are selected from the group consisting of poly(ethylene oxide), polyvinyl pyrrolidone, Dextran, saccharide, cellulose, chitosan, gelatin, collagen, polyvinyl alcohol, Eudragit, polyethylene terephthalate, polyester, polymethylmethacrylate, polyacrylonitrile, silicone, polyurethane, polycarbonate, polyether ketone ketone, polyether ether ketone, polyether imide, polyamide, polystyrene, polyether sulfone, polysulfone, polycaprolactone, polylactic acid, polylactide-co-caprolactone, polylactide-co-glycolide, polyglycolic acid, polyglycerol sebacic, polydiol citrate, polyhydroxy butyrate, polyether amide, polydioxanone, derivatives thereof, and combinations thereof.

10. The electrospun structure of claim 1, wherein the electrospun structure is in the form of a sheet.

11. The electrospun structure of claim 10, wherein the sheet has an average length of about 1 cm to about 6 cm, an average width of about 5 mm to about 10 mm, and an average thickness of about 1 mm to about 2 mm.

12. A method of fabricating an electrospun structure, the method comprising:
dispersing a pharmaceutical into a polymer solution comprising one or more polymers;
electrospinning the polymer solution to form the electrospun structure comprising the pharmaceutical dispersed throughout the one or more electrospun polymers of the electrospun structure;
wherein the pharmaceutical comprises at least one of a cannabinoid, a phytocannabinoid, a terpene produced by a Cannabis plant, or a combination thereof;
wherein the one or more electrospun polymers are configured to degrade over a predetermined time period to deliver the pharmaceutical over the predetermined time period.

13. The method of claim 12, wherein the pharmaceutical is present in an amount of at least 100 wt % relative to the one or more polymers.

14. The method of claim 12, wherein the one or more polymers comprise a first polymer and a second polymer.

15. The method of claim 14, wherein electrospinning the polymer solution comprises co-electrospinning a first polymer solution comprising the first polymer and a second polymer solution comprising the second polymer to form the electrospun structure.

16. The method of claim 14, wherein:
the first polymer is configured to degrade over a first time period to release the pharmaceutical over the first time period and the second polymer is configured to degrade over a second time period to release the pharmaceutical over the second time period.

17. The method of claim 16, wherein the first time period and the second time period do not overlap.

18. The method of claim 16, wherein the first time period and the second time period at least partially overlap.

19. The method of claim 14, wherein the first polymer and the second polymer are arranged in a coaxial configuration.

20. The method of claim 12, wherein the one or more polymers are selected from the group consisting of poly(ethylene oxide), polyvinyl pyrrolidone, Dextran, saccharide, cellulose, chitosan, gelatin, collagen, polyvinyl alcohol, Eudragit, polyethylene terephthalate, polyester, polymethylmethacrylate, polyacrylonitrile, silicone, polyurethane, polycarbonate, polyether ketone ketone, polyether ether ketone, polyether imide, polyamide, polystyrene, polyether sulfone, polysulfone, polycaprolactone, polylactic acid, polylactide-co-caprolactone, polylactide-co-glycolide, polyglycolic acid, polyglycerol sebacic, polydiol citrate, polyhydroxy butyrate, polyether amide, polydioxanone, derivatives thereof, and combinations thereof.

21. The method of claim 12, wherein the electrospun structure is in the form of a sheet.

22. The method of claim 21, wherein the sheet has an average length of about 1 cm to about 6 cm, an average width of about 5 mm to about 10 mm, and an average thickness of about 1 mm to about 2 mm.
